(19) United States
(12) Patent Application Publication
(10) Pub. No.: US 2005/0095303 A1
(43) Pub. Date: May 5, 2005

(54) Highly purity bioactive glass and method for the production thereof

(76) Inventors: Stephen Krenitski, Old Forge, PA (US); Kiefer Werner, Mainz (DE); Sybill Nuttgens, Frankfurt (DE); Michael Leister, Budenheim (DE); Volker Ohmstede, Mainz (DE); Uwe Kolberg, Mainz (DE); Roland Schnabel, Hofheim (DE)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 10/491,578

(22) PCT Filed: Oct. 2, 2002
(86) PCT No.: PCT/EP02/11007

(30) Foreign Application Priority Data
Oct. 2, 2001 (DE) 101 49 309.6

(51) Int. Cl. C03C 3/097; A61K 33/42

(52) U.S. Cl. 424/604; 501/63

(57) ABSTRACT

The present invention relates to a high-purity bioactive glass, having the following composition in % by weight:

| | |
|---|---|
| SiO₂ | 35-86 |
| Na₂O | 5.5-35 |
| CaO | 4-46 |
| P₂O₅ | 1-15 |
| Further additional | 0.05-15 |
| substances | |

and to a process for producing it, in which the glass is produced in a radiofrequency-heated skull crucible.

The invention relates to a high-purity bioactive glass, and to a process for producing it.

The term bioactive or biocompatible materials is to be understood as meaning materials which are biologically tolerable in a biological environment, such as bones, joints, teeth or alternatively skin or hair, and functionally match themselves to their surroundings. Bioactive materials also encompass bioactive glasses, which generally have a composition in % by weight of:

| | |
|---|---|
| SiO₂ | 40-86 |
| Na₂O | 0-35 |
| CaO | 4-46 |
| P₂O₅ | 1-15 |

Bioactive glasses of this type are described, for example, in ‘An Introduction to Bioceramics’, L. Hench and J. Wilson, eds. World Scientific, New Jersey (1993).

For many applications in the medical and cosmetic sector, it is preferable to use bioactive glasses which have a high alkali metal content. These glasses achieve various effects, such as an antimicrobial action, a solubility which is set in an aqueous environment and can be adjusted by means of the other glass components, such as additional multivalent metal ions, or repolymerization of the polysilicic acid at the surface at a weakly alkaline pH. Glasses having these actions generally have the following composition (in % by weight):

| | |
|---|---|
| SiO₂ | 40-68 |
| Na₂O | 5-30 |
| CaO | 10-35 |
| P₂O₅ | 1-12 |

In addition, or alternatively as an exchange for individual components, depending on the particular application, it is also possible for further components, such as CaF₂, B₂O₃, Al₂O₃, MgO or K₂O, to be present, generally in amounts of between 0 and 10% by weight.

By way of example, a known bioactive glass has a composition (in % by weight) of

| | |
|---|---|
| SiO₂ | 45 |
| Na₂O | 24.5 |
| CaO | 24.5 |
| P₂O₅ | 6 |

In these biologically active glasses, the solubility or breaking-up of the SiO₂ network is based on the Na₂O and CaO contents which are set, with the high bioactivity being based on the high Cao and P₂O₅ contents, leading to the formation of a layer of hydroxycarbonate apatite. This layer promotes the interaction with the biological environment.

Bioactive glasses are normally produced and used in powder form, with the mean particle size (measured using light-scattering methods) preferably being <90 μm, in special cases <20 μm and particularly preferably <5 μm. As the particle size decreases, the active specific surface area of the powder increases, so that in this way it is also possible to control the degree of interaction.

Glasses of this type are produced using a discontinuous melting process at melting temperatures of between 1250° C. and 1400° C., generally from oxides or carbonate compounds as starting materials.

The production is described as follows in U.S. Pat. No. 6,051,247 and WO 94/04657. The starting materials (SiO₂, Na₂O, P₂O₅, CaO) are mixed in a plastic container in a ball mill for 4 hours. The mixture produced is then melted in a platinum crucible at 1350° C. and homogenized for 24 h. The melted glass is then poured into distilled, deionized water in order to obtain a glass frit. The frit is then comminuted in a mortar using a pestle and screened by means of ASTM screening in order to produce the required particle size distribution.

These melting processes involve serious drawbacks in particular for a bioactive glass. The corrosive behavior of the bioactive glasses of the compositions listed leads to extensive dissolution of the platinum in the melting crucible, and platinum particles may enter the glass. Platinum may lead to undesirable side effects in particular for bioactive applications.

The discontinuous melting process, in particular in the case of glasses with components which can evaporate, such as for example alkali metals, leads not only to shifts in the composition but also inhomogeneities within the melting crucible. Since the effectiveness of the bioactive glasses is significantly dependent on the constancy of composition and the ratio of the Na₂O/CaO and CaO/P₂O₅ contents, shifts within the set contents cannot be tolerated.

A discontinuous crucible melting is undesirable for industrial production if a continuous production process without fluctuations in composition is the aim.

The object of the invention is to provide a bioactive glass which has the purity required for the particular biological applications.

The object is achieved by a high-purity bioactive glass, having the following composition in % by weight:

| | |
|---|---|
| SiO₂ | 35-86 |
| Na₂O | 5.5-35 |
| CaO | 4-46 |
| P₂O₅ | 1-15 |
| Further additional | 0.05-15 |
| substances | |

with the glass being produced in a radiofrequency-heated skull crucible.

The object is also achieved by the features of claims 2 to 13.

On account of their extremely aggressive nature, the bioactive glasses cannot be melted in a continuous and stable melting process and with the required purity using conventional melting methods.

The refractory materials made from Al₂O₃ or ZrO₂ which are used for melting technical-grade glasses, and also the platinum or quartz melting vessels used to melt optical glasses, are not suitable for long-term and therefore stable production of high-purity bioactive glasses.

Ceramic refractory materials are generally used to melt glasses. Refractory ceramics formed from Al₂O₃ and ZrO₂ have proven particularly suitable. These refractory materials are attacked and corroded very strongly by the bioactive glasses, which contain SiO₂, Na₂O, CaO and P₂O₅.

For many applications of bioactive glasses, the aluminum or zirconium content must not exceed defined limits. However, these limits are generally exceeded as a result of the extensive corrosion of the melting crucibles.

The crucible is rendered unusable by the strong attack from the bioactive glass after just a few days, since it has been completely corroded through. Crucibles made from these refractory materials can only be used for extremely short melting periods or discontinuous melting with subsequent reconstruction.

Bioactive glasses are so aggressive with respect to melting units made from platinum or platinum alloys that the melted glasses either acquire a gray tinge from the dissolved platinum metal or acquire a strong yellow tinge from the dissolved platinum ions, if the melting is carried out in a strongly oxidizing atmosphere. For some applications, the high platinum content in the bioactive glasses may cause problems, since it is known from chemistry that platinum acts as a catalyst for many chemical reactions. Furthermore, the high degree of platinum corrosion leads to extensive corrosion of the platinum crucible even after just a very short time. Further melting is impossible for safety reasons. In addition to the constantly high refitting and failure costs, a further factor is the very high cost of platinum and the restoring of the platinum apparatus.

It is preferable for melting crucibles made from quartz material to be used to produce high-purity optical glasses. It has been found that bioglasses of the composition listed above also attack the quartz material so strongly that the quartz crucible has been dissolved after just a few hours or at most days. Since the SiO₂ dissolves in the glass melt, a glass of constant composition can only be produced with difficulty. Even with crucibles made from quartz material, it is only possible for extremely short melting periods or even only discontinuous melting operations, with the associated high melting costs, to be carried out.

According to the invention, bioactive glasses, despite their extremely aggressive nature, can be produced in a stable melting process and in high-purity form.

Melting of glasses and crystals using radiofrequency in a skull crucible is used primarily for high-melting crystals, such as ZrO₂, or high-melting glasses. A skull comprising the crystal or glass which is to be melted is formed on the water-cooled metal tubes which form the skull crucibles. In the case of high-melting crystals, such as ZrO₂, a relatively thick skull layer of weakly sintered powder of ZrO₂ crystals is formed. Even high-melting glasses still form a relatively thick skull layer. In the case of low-melting glasses, this skull layer becomes thinner, and the risk of the melt reacting with the metal tubes of the skull crucible becomes ever greater.

It is therefore to be expected that, in the case of the extremely aggressive bioactive glasses, the thin skull layer will entail corrosion and therefore destruction of the skull crucibles.

Surprisingly, however, it has been discovered that the aggressive glass melt of the bioactive glasses can attack the metal tubes which form the skull crucible through the skull layer. This attack does not generally lead to destruction of the metal tubes, but rather may even be used to enrich the glass melt in a targeted fashion. This makes it possible, for example, to achieve a desired blue coloration or antimicrobial action.

Unlike in the case of the very high-melting crystals, in the case of glasses sparkovers may occur in the glass melt, and these can likewise destroy the skull crucibles. However, these sparkovers can be avoided if the metal tubes which form the skull crucible are short-circuited in the region of the radiofrequency field.

The water-cooled metal tubes of the skull crucible used are generally copper tubes. The extremely aggressive bioactive glass attacks the copper tube through the skull layer and imparts a green or blue color to the glass, depending on the oxidation state of the glass. The quantity of copper which has diffused into the bioactive glass is very small, in the ppm range. For example, 2 ppm were measured in a melted bioactive glass. For some applications, coloration of the glass is unacceptable. For other applications, the copper ions may be disruptive. However, in certain cases, since copper is antibacterial, it may be tolerated or may even be desirable. The use of the copper tubes as skull material is therefore highly dependent on the subsequent use of the melted bioactive glass.

However, the extent to which the bioactive glasses attack the copper tubes of the skull crucible is not so great that the corrosion leads to destruction of the tubes during production. Therefore, copper tubes, taking account of the restrictions relating to the purity of the glass melt, are suitable for the production of bioactive glasses.

In addition to the skull crucible made from copper tubes, skull crucibles made from special steel tubes have also been tested. The coloration of the bioactive glasses is significantly reduced in the case of special steel tubes being used. The quantities of dissolved CoO and Cr₂O₃ are less than 1 ppm, and the quantity of dissolved Nio is less than 5 ppm, below the respective detection limits for the analysis methods employed. The quantity of Fe₂O₃ which is dissolved out of the special steel tubes is well below the quantity of Fe₂O₃ which is introduced by the batch.

Skull crucibles formed from platinum tubes have also been tested. Unlike with the melts which were formed in platinum crucibles, in the case of skull crucible melting it was impossible to detect any contamination of the glass melt or corrosion to the platinum tubes. Since platinum is more noble than special steel and copper, the attack of the bioglasses on the platinum is still not as strong as on the latter materials.

If there are very strict demands relating to heavy metals in the bioactive glasses, it is also possible to use a skull crucible made from aluminum tubes. It is impossible to detect any additional aluminum above the quantity of aluminum which is introduced by the raw materials in the melted bioactive glasses.

For ultra-high-purity requirements, a skull crucible whose water-cooled metal tubes were covered with plastic has been tested. These tubes are not attacked by the bioactive glasses. There was no evidence of any change to the glass melt or of corrosion to the plastic-coated metal tubes.

The tests carried out demonstrate that it is possible to melt the extremely aggressive bioactive glasses in radiofrequency-heated skull crucibles. To ensure that the different purity requirements imposed on the various bioactive glasses are complied with, the invention provides skull crucibles with metal tubes made from different materials.

To make it possible to melt glasses using radiofrequency, the glasses must have a sufficient electrical conductivity to enable them to be coupled to radiofrequency. The quantity of energy which is introduced into the glass melt by the radiofrequency must be greater than the quantity of heat which is extracted from the glass melt as a result of heat being radiated out of the surface or as a result of heat being dissipated through the water-cooled metal tubes. Therefore, in addition to the electrical conductivity of the glasses, other factors also play an important role in connection with radiofrequency melting in skull crucibles, such as for example the geometry, volume or structure of the melting crucible and the materials used for the metal tubes of the skull crucibles.

For example, it has been found that the skull crucibles having the various metal tubes have different energy demands for the melting of the glass. Under identical conditions, the copper skull and the aluminum skull, at 9 kW and 7 kW, have a lower generator power loss than the special steel skull or the plastic-coated special steel skull, which are significantly worse, with generator power losses of 15 kW and 14 kW for the same dimensions of skull crucible.

Particularly in the case of batches which are very difficult to melt, it is important to achieve the highest possible generator powers. If the purity requirements allow, therefore, skull crucibles made from copper tubes are preferred. Skull crucibles made from aluminum tubes have the same low power losses and are in most cases better in terms of purity. However, they have the drawback of being very difficult to produce.

As has already been mentioned, glasses have to have a sufficient electrical conductivity at the melting temperature to enable them to be melted using radiofrequency. Not all bioactive glasses satisfy this requirement, but rather only the glasses according to the invention do so.

The electrical conductivity of the bioactive glasses is substantially determined by the alkali metal content, i.e. by the Na₂O content.

Bioactive glasses can also be used as glass with an antimicrobial action. These glasses preferably contain silver and/or copper ions. However, they may also contain other ions, such as zinc, tin, bismuth, cerium, nickel or cobalt or combinations of these ions. These ions may in each case be present in amounts of between 0.5 and 15.0% by weight.

The electrical conductivity of the bioactive glasses is increased by the monovalent ions of silver and copper. Both elements are comparable to sodium in terms of electrical conductivity. The sum of Na₂O, Ag₂O and Cu₂O is preferably greater than or equal to 6%. With this composition, the glass can be melted using radiofrequency. The divalent ions likewise contribute to increasing the electrical conductivity, but to a significantly lesser extent.

Various compositions of the bioactive glass described above were melted in order to specifically determine the glass compositions which can be produced by means of the RF technology. A crucible which is surrounded by an RF coil and is heated by an RF generator was used. The compositions of the glasses melted using the RF technique are shown in the table below; both a melt without any Na₂O and a melt containing just 5% by weight of Na₂O were not sufficiently coupled to the RF field, and therefore the conductivity of these glasses is insufficient to allow the required quantity of heat to be introduced into the glass using the RF technology.

The following results of the tests aimed at restricting the composition range were obtained. The composition: 33% by weight of CaO; 9% by weight of P₂O₅ and 58% by weight of SiO₂ cannot be melted using radiofrequency.

| Batch | | | | | |
|---|---|---|---|---|---|
| Na₂O | SiO₂ | CaO | P₂O₅ | | |
| [% by | [% by | [% by | [% by | Coupling | |
| weight] | weight] | weight] | weight] | performance | Melt |
| 11.5 | 58 | 24.5 | 6.0 | RF coupling | S1 |
| | | | | achieved | |
| 8 | 61.5 | 24.5 | 6.0 | RE coupling | S2 |
| | | | | achieved | |
| 6.6 | 62.8 | 24.6 | 6.0 | RE coupling | S3 |
| | | | | achieved | |
| 6.6 | 55.7 | 30.3 | 7.4 | RE coupling | S4 |
| | | | | achieved | |
| 5.1 | 64.3 | 24.6 | 6.0 | RE coupling | S5 |
| | | | | not achieved | |
| 0 | 58 | 33 | 9 | RE coupling | S6 |
| | | | | not achieved | |

The inventors have surprisingly discovered that not only is the Na₂O content in the melt important for the coupling performance, but also a Na₂O+P₂O₅/SiO₂ ratio best reflects the coupling performance of the glass. The table below shows the melts in order of coupling performance, together with the details of the Na₂O+P₂O₅/SiO₂ ratio.

| | Na₂O + P₂O₅/SiO₂ |
|---|---|
| RF coupling | ratio |
| S1 (very good) | 0.30 |
| S4 | 0.25 |
| S2 | 0.22 |
| S3 | 0.20 |
| S5 (none) | 0.17 |
| S6 (none) | 0.16 |

It is clear from these results that, to achieve sufficient RF coupling to the melt, the Na₂O+P₂O₅/SiO₂ ratio must be at least 0.18.

The conductivity required for the glasses for melting in an RF melting installation may differ for different installations. The constancy of the composition of the bioactive glasses depends to a significant degree on whether there was any dusting of the batch during the initial melting or whether glass constituents evaporated out of the glass surface during the melting operation. On account of the high purity required, synthetic raw materials generally have to be used for the bioactive glasses, and such raw materials in some cases have a considerable tendency to dusting.

In a comparative test, a dusting rate of 1.04 g/h per standardized unit was found for the composition: Na₂O: 24.5% by weight, CaO 24.5% by weight; P₂O₅ 6.0% by weight; SiO₂ 45.0% by weight, using batch 1 comprising sodium hydrogen carbonate, calcium carbonate, monocalcium phosphate and silica flour. With batch 2, lime (produced for optical glasses) was used instead of calcium carbonate, and sodium metaphosphate was used instead of monocalcium phosphate, making it possible to reduce the dusting to 0.48 g/h per standardized unit area.

In addition to the purity of the glass melt and the constancy of the composition, the economics of glassmaking also play an important role.

According to the invention, the bioactive glasses can be produced both discontinuously and continuously, since the attack on the skull crucibles by the bioactive glasses is so is minor that the service life of the crucibles is not affected by the corrosion. If the bioactive glass is milled to form glass powder in the subsequent process, the glass melt does not need to be refined. In a discontinuous melting process, the glass melt, after it has been melted down, can be poured out through a bottom outlet. The glass melt, after it has been melted down, does not have to be subjected to any additional homogenization process, since the glass melt is homogenized very thoroughly by the very strong convection prevailing in the skull crucible.

For continuous melting, according to the invention it has proven particularly advantageous to carry out the glass melting in the skull crucible in which the melting area is divided by a bridge formed from water-cooled metal tubes, with the bridge only projecting into the upper part of the glass melt. Surprisingly, it has been found that the batch, which is laid onto the melt on one half, is initially drawn downward by the convection and in the process is rapidly melted down, before then rising up in the other half, where the glass is drawn off at the top.

To further improve the throughput, according to the invention it is possible for the melting-down process to be accelerated by introducing a gas into the glass melt from below. In the case of the skull crucible which is divided by a bridge, the bubbling gas is introduced into that part into which the batch is laid. Bubbling with a gas, such as for example an O₂ gas, an inert gas such as N₂ gas or a noble gas, such as He or Ar gas, makes it possible to increase the melting-down performance by a factor of ≧2.

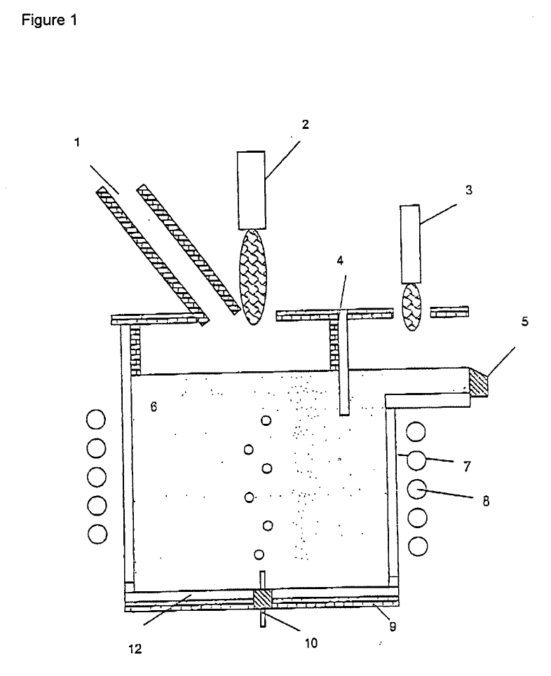

The invention is explained in more detail below with reference to a drawing. The drawing comprises FIG. 1. FIG. 1 shows the structure of a skull crucible.

What is shown in detail is an introduction opening (1), a tank furnace burner (2), an overflow burner (quartz glass) (3), a bridge (4), an outlet (5), a melt (6), a skull crucible (7), an RF coil (8), Quarzal base plate (9), bubbling nozzle (10) and a cooled base plate (11).

1. A high-purity bioactive glass, comprising:
SiO₂ in the range of 35-86 based on a percent per weight of the total composition;
Na₂O in the range of 5.5-35 based on a percent per weight of the total composition;
CaO in the range of 4-46 based on a percent per weight of the total composition;
P₂O₅ in the range of 1-15 based on a percent per weight of the total composition; and
one or more additional substances having a total percent per weight of the total composition in the range of 0.05-15, wherein the high-purity bioactive glass is produced in a radio_frequency-heated skull crucible and has a ratio of Na₂O+P₂O₅ to SiO₂ of at least 0.18.

2. The high-purity bioactive glass as claimed in claim 1, wherein the SiO₂ has a percent per weight of the total composition in the range of 40-86 and the Na₂O has a percent per weight of the total composition in the range of 6.5-35.

3. The high-purity bioactive glass as claimed in claim 1, wherein the one or more additional substances have one or more substances selected from the group consisting of Ag₂O, Cu₂O, CuO, ZnO, SnO, Bi₂O₃, Ce₂O₃, NiO, CoO, and any combinations thereof.

4. The high-purity bioactive glass as claimed in claim 3, wherein the sum of Na₂O, Ag₂O and Cu₂O is greater than or equal to 6% by weight.

5. The high-purity bioactive glass as claimed in claim 1, wherein the high-purity bioactive glass is produced in a continuous melting process.

6. The high-purity bioactive glass as claimed in claim 1, wherein the high-purity bioactive glass is produced in a discontinuous melting process.

7. The high-purity bioactive glass as claimed in claim 1, wherein the radio frequency-heated skull crucible has water-cooled metal tubes made of a material selected from the group consisting of copper, special steel, platinum metal, platinum alloy, and aluminum metal.

8. The high-purity bioactive glass as claimed in claim 7, wherein the water-cooled metal tubes have plastic-coated metal tubes.

9. The high-purity bioactive glass as claimed in claim 1, wherein the high-purity bioactive glass is taken off at a glass outlet positioned at the top of the radio frequency-heated skull crucible, and in which a water-cooled, metallic bridge is immersed in a melt of the high-purity bioactive glass and separates a batch area of the radio frequency-heated skull crucible from the glass outlet.

10. The high-purity bioactive glass as claimed in claim 9, wherein the degree of mixing in the batch area is additionally increased by bubbling.

11. A process for producing a high-purity bioactive glass comprising:
adding a plurality of glass components to a radio frequency-heated skull crucible, the plurality of glass components comprising SiO₂ in a range of 35 to 86 based on a percent weight of the total composition, Na₂O in a range of 5.5 to 35 based on a percent weight of the total composition, CaO in a range of 4 to 46 based on a percent weight of the total composition, P₂O₅ in a range of 1 to 15 based on a percent weight of the total composition, and additional substances in a range of 0.05 to 15 based on a percent weight of the total composition, wherein a ratio of Na₂O+P₂O₅ to SiO₂ is at least 0.18; and
controlling the radio frequency-heated skull crucible to form a glass melt from the plurality of glass components.

12. The process for producing the high-purity bioactive glass as claimed in claim 11, wherein the glass melt has a homogenous and constant composition.

13. The process for producing the high-purity bioactive glass as claimed in claim 11, wherein the process is a continuous melting process or a discontinuous melting process.

14. The process for producing the high-purity bioactive glass as claimed in claim 11, further comprising taking the glass melt from a glass outlet at a top of the radio frequency-heated skull crucible.

15. The process for producing the high-purity bioactive glass as claimed in claim 14, further comprising immersing a water-cooled, metallic bridge in the glass melt to define a batch area and the glass outlet.

16. The process for producing the high-purity bioactive glass as claimed in claim 15, further comprising introducing bubbles to the glass melt in the batch area to mix the glass melt.

17. A high-purity bioactive glass, comprising:
SiO₂ in a range of 35 to 86 based on a percent weight of the total composition;
Na₂O in a range of 5.5 to 35 based on a percent weight of the total composition;
CaO in a range of 4 to 46 based on a percent weight of the total composition; and
P₂O₅ in a range of 1 to 15 based on a percent weight of the total composition, wherein a ratio of Na₂O+P₂O₅ to SiO₂ is at least 0.18.

18. The high-purity bioactive glass as claimed in claim 17, further comprising one or more additional substances in a range of 0.05 to 15 based on a percent weight of the total composition.

19. The high-purity bioactive glass as claimed in claim 18, wherein the one or more additional substances is at least one substance selected from the group consisting of Ag₂O, Cu₂O, CuO, ZnO, SnO, Bi₂O₃, Ce₂O₃, NiO, CoO, and any combinations thereof.

20. The high-purity bioactive glass as claimed in claim 19, wherein the sum of Na₂O, Ag₂O and Cu₂O is greater than or equal to 6 percent weight of the total composition.
